(19) United States
(12) Patent Application Publication
(10) Pub. No.: US 2009/0024031 A1
(43) Pub. Date: Jan. 22, 2009

(54) ULTRASONIC DIAGNOSTIC APPARATUS AND ULTRASONIC DIAGNOSTIC METHOD

(75) Inventors: Hiroyuki Ohuchi, Nasushiobara-shi (JP); Tetsuya Kawagishi, Nasushiobara-shi (JP); Tomohisa Imamura, Nasushiobara-shi (JP)

Correspondence Address: OBLON, SPIVAK, MCCLELLAND MAIER & NEUSTADT, P.C., 1940 DUKE STREET, ALEXANDRIA, VA, 22314, US

(73) Assignees: KABUSHIKI KAISHA TOSHIBA, Tokyo (JP); TOSHIBA MEDICAL SYSTEMS CORPORATION, Otawara-Shi (JP)

(21) Appl. No.: 12/173,321

(22) Filed: Jul. 15, 2008

(30) Foreign Application Priority Data
Jul. 18, 2007 (JP) 2007-187407

(51) Int. Cl. A61B 8/00

(52) U.S. Cl. 600/440

(57) ABSTRACT

An ultrasonic diagnostic apparatus includes an ultrasonic transmission/reception unit and an image generating unit. The ultrasonic transmission/reception unit transmits a first ultrasonic pulse having a maximum amplitude in a positive pressure, a second ultrasonic pulse having a maximum amplitude in a negative pressure and a third ultrasonic pulse derived by compounding the first ultrasonic pulse with the second ultrasonic pulse into an object in which a contrast medium is injected and obtains first, second and third reception echoes corresponding to the first, second and third ultrasonic pulses respectively. The image generating unit generates a compounded signal which is a nonlinear component from the contrast medium by a linear calculation of the first, second and third reception echoes and generates image data of the object by using the compounded signal.

### BACKGROUND OF THE INVENTION

1. Field of the Invention

The present invention relates to an ultrasonic diagnostic apparatus and an ultrasonic diagnostic method that obtain biological information of an object by irradiating ultrasonic pulses into the object, receiving ultrasonic echoes generated in the object, and performing various processing to the received ultrasonic echoes, in particular, to an ultrasonic diagnostic apparatus and an ultrasonic diagnostic method which can perform imaging according to a contrast echo method with using a contrast medium.

2. Description of the Related Art

An ultrasonic diagnostic apparatus is an apparatus that obtains biological information such as tomographic images of living body tissues and blood flow images in an object, by irradiating ultrasonic pulses into the object from piezoelectric transducers (ultrasonic transducers) built in an ultrasonic probe, receiving ultrasonic echoes generated in the object with the piezoelectric transducers, and performing various processing on the received ultrasonic echoes.

An imaging method employed by such ultrasonic diagnostic apparatuses is an imaging method referred to as a contrast echo method. In the contrast echo method, microbubbles are injected into a blood vessel of the object as a contrast medium to enhance the scattering ultrasonic echoes. More specifically, in imaging according to the contrast echo method, ultrasonic pulses having predetermined frequency spectra are irradiated, and nonlinear components of the ultrasonic echoes reflected from the microbubbles as the contrast medium are used for visualization (see, for example, Japanese Patent Application (Laid-Open) No. 8-182680).

In this contrast echo method, there has been a report that micro bubbles respond more strongly, i.e., generate an ultrasonic echo having nonlinear component with a higher signal intensity to an ultrasonic wave having an amplitude in the negative pressure side than an ultrasonic wave having an amplitude in the positive pressure side (for example, refer to a document “Experimental and Theoretical Evaluation of Microbubble Behavior: Effect of Transmitted Phase and Bubble Size”, Karen E. Morgan, John S. Allen, Paul A. Dayton, James E. Chomas, Alexander L. Klibanov and Katherine W. Ferrara, Member, IEEE, IEEE TRANSACTIONS ON ULTRASONICS, FERROELECTRICS, AND FREQUENCY CONTROL, VOL. 47, No. 6, NOVEMBER 2000).

Additionally in the contrast echo method, a technique has been devised that second harmonic component included in an ultrasonic echo from micro bubbles is used for imaging. In this technique, nonlinear component included in second harmonic of an ultrasonic echo is extracted, and a contrast-enhanced image of micro bubbles is obtained using the extracted nonlinear component.

However, in the conventional contrast echo method using second harmonic component, the second harmonic component contains not only a tissue echo that is linear component from a living body tissue but also tissue harmonic component that is a tissue echo of nonlinear component. Therefore, there is a problem that a tissue echo cannot be completely eliminated. As a result, the conventional contrast echo method using second harmonic component cannot keep the contrast ratio sufficiently in an ultrasonic diagnostic image, that is, the intensity ratio of an ultrasonic echo signal from micro bubbles to a tissue echo signal.

### SUMMARY OF THE INVENTION

The present invention has been made in light of the conventional situations, and it is an object of the present invention to provide an ultrasonic diagnostic apparatus and an ultrasonic diagnostic method which make it possible to obtain an ultrasonic diagnostic image with a more satisfactory contrast ratio by imaging under the contrast echo method performed with injecting a contrast medium into an object.

The present invention provides an ultrasonic diagnostic apparatus comprising: an ultrasonic transmission/reception unit configured to transmit a first ultrasonic pulse having a maximum amplitude in a positive pressure, a second ultrasonic pulse having a maximum amplitude in a negative pressure and a third ultrasonic pulse derived by compounding the first ultrasonic pulse with the second ultrasonic pulse into an object in which a contrast medium is injected and obtain a first reception echo, a second reception echo and a third reception echo corresponding to the first ultrasonic pulse, the second ultrasonic pulse and the third ultrasonic pulse respectively; and an image generating unit configured to generate a compounded signal which is a nonlinear component from the contrast medium by a linear calculation of the first reception echo, the second reception echo and the third reception echo and generate image data of the object by using the compounded signal, in an aspect to achieve the object.

The present invention also provides an ultrasonic diagnostic method comprising: transmitting a first ultrasonic pulse having a maximum amplitude in a positive pressure, a second ultrasonic pulse having a maximum amplitude in a negative pressure and a third ultrasonic pulse derived by compounding the first ultrasonic pulse with the second ultrasonic pulse into an object in which a contrast medium is injected and obtaining a first reception echo, a second reception echo and a third reception echo corresponding to the first ultrasonic pulse, the second ultrasonic pulse and the third ultrasonic pulse respectively; and generating a compounded signal which is a nonlinear component from the contrast medium by a linear calculation of the first reception echo, the second reception echo and the third reception echo and generating image data of the object by using the compounded signal, in an aspect to achieve the object.

The ultrasonic diagnostic apparatus and the ultrasonic diagnostic method as described above make it possible to obtain an ultrasonic diagnostic image with a more satisfactory contrast ratio by imaging under the contrast echo method performed with injecting a contrast medium into an object.

### BRIEF DESCRIPTION OF THE DRAWINGS

In the accompanying drawings:

FIG. 1 is a block diagram of an ultrasonic diagnostic apparatus according to an embodiment of the present invention;

FIG. 2 is a flowchart showing a procedure for obtaining an ultrasonic diagnostic image of an object according to the contrast echo method using microbubbles as a contrast medium by the ultrasonic diagnostic apparatus shown in FIG. 1;

FIG. 3 is a diagram showing an example of setting the first ultrasonic pulse, the second ultrasonic pulse and the third ultrasonic pulse transmitted toward the object from the ultrasonic diagnostic apparatus shown in FIG. 1 for a simulation;

FIG. 4 is a diagram showing an example of a frequency spectrum of the first ultrasonic pulse, the second ultrasonic pulse and the third ultrasonic pulse each shown in FIG. 3;

FIG. 5 is a diagram showing a compound signal derived by a simulation using the first ultrasonic pulse, the second ultrasonic pulse and the third ultrasonic pulse shown in FIG. 3;

FIG. 6 is a diagram showing each frequency spectrum of the first reception echo, the second reception echo and the third reception echo shown in FIG. 5 (C);

FIG. 7 is a diagram showing the frequency spectrum of the compound signal shown in FIG. 5 (D);

FIG. 8 is a diagram showing an example case of transmitting the first ultrasonic pulse, the second ultrasonic pulse and the third ultrasonic pulse from the ultrasonic diagnostic apparatus shown in FIG. 1 with setting the amplitudes, the phases, the center frequencies and the frequency bands thereof into desired values;

FIG. 9 is a diagram showing channels formed by the plural ultrasonic transducers included in the ultrasonic probe shown in FIG. 1; and

FIG. 10 is a diagram showing an example case of synthesizing the third ultrasonic pulses as a transmission acoustic field by transmitting the first ultrasonic pulses and the second ultrasonic pulses from mutually different and exclusive plural channels of the ultrasonic probe shown in FIG. 9.

### DESCRIPTION OF THE PREFERRED EMBODIMENTS

An ultrasonic diagnostic apparatus and an ultrasonic diagnostic method according to embodiments of the present invention will be described with reference to the accompanying drawings.

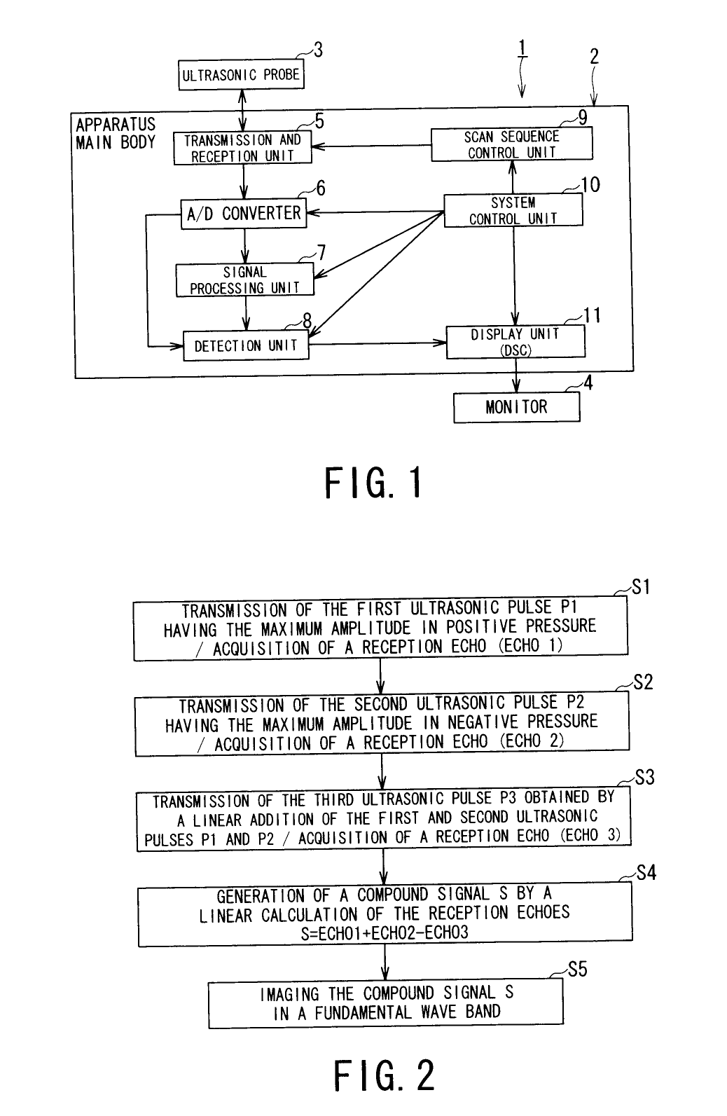

FIG. 1 is a block diagram of an ultrasonic diagnostic apparatus according to an embodiment of the present invention.

An ultrasonic diagnostic apparatus 1 includes an apparatus main body 2 provided with an ultrasonic probe 3 and a monitor 4. The apparatus main body 2 has a transmission and reception unit 5, an A/D (analog to digital) converter 6, a signal processing unit 7, a detection unit 8, a scan sequence control unit 9, a system control unit 10, and a display unit 11. Each element in the apparatus main body 2 may be configured by circuits or by a CPU (central processing unit) of a computer loading a control program.

The ultrasonic probe 3 has a plurality of ultrasonic transducers. Each ultrasonic transducer has a function to transmit an ultrasonic pulse into an object (not shown) after converting a transmission signal, applied by the transmission and reception unit 5 as an electric pulse, into the ultrasonic pulse. In addition, each of the ultrasonic transducers has a function to receive an ultrasonic echo caused by the ultrasonic pulse having transmitted into the object and supply the received ultrasonic echo to the transmission and reception unit 5 as a reception echo which is an electronic signal. That is, the ultrasonic probe 3 is configured to transmit an ultrasonic pulse from each of the plural transmission channels corresponding to the ultrasonic transducers respectively and receive an ultrasonic echo by each of the plural reception channels.

The transmission and reception unit 5 has a function to supply a transmission signal to each ultrasonic transducer of the ultrasonic probe 3 according to a control signal supplied from the scan sequence control unit 9 as a scan sequence, thereby controlling the ultrasonic probe 3 so that ultrasonic pulses having predetermined characteristics are transmitted from the ultrasonic probe 3. In addition, the transmission and reception unit 5 has a function to receive reception echoes from the ultrasonic probe 3, to perform predetermined preprocessing, such as delay processing and phasing addition processing, and to supply the processed reception echoes to the A/D converter 6.

The scan sequence control unit 9 has a function to control the transmission and reception unit 5 by supplying a control signal to the transmission and reception unit 5 as a scan sequence so that ultrasonic pulses having predetermined waveforms are transmitted from the ultrasonic probe 3. More specifically, the scan sequence control unit 9 has a function to control the transmission and reception unit 5 by supplying a control signal to the transmission and reception unit 5 so that the first ultrasonic pulse having the maximum amplitude (peak) in positive pressure, the second ultrasonic pulse having the maximum amplitude in negative pressure and the third ultrasonic pulse derived by compounding the first ultrasonic pulse with the second ultrasonic pulse are transmitted from the ultrasonic probe 3 sequentially in an arbitrary order. In addition, the scan sequence control unit 9 is configured to control an amplitude, a phase, a center frequency and a frequency band of each of the first ultrasonic pulse, the second ultrasonic pulse and the third ultrasonic pulse into desired values respectively, as needed. Further, the scan sequence control unit 9 is also equipped with the function to shift time phases of the respective maximum amplitudes of the first ultrasonic pulse, the second ultrasonic pulse and the third ultrasonic pulse mutually to transmit them sequentially.

The A/D converter 6 has a function to supply digital reception echoes to the signal processing unit 7 or the detection unit 8 after digitalizing analog reception echoes received from the transmission and reception unit 5.

The signal processing unit 7 has a function to perform signal processing on the reception echoes received from the A/D converter 6. The signal processing unit 7 also has a function to supply a compound signal obtained by the signal processing to the detection unit 8. More specifically, the signal processing unit 7 is configured to perform the signal processing for combining the first reception echo, the second reception echo and the third reception echo respectively corresponding to the first ultrasonic pulse, the second ultrasonic pulse and the third ultrasonic pulse by a linear calculation to generate a compound signal. In the compounding above-described, the signal processing unit 7 is configured to correct phases and/or amplitudes of the first reception echo, the second reception echo and the third reception echo, as needed.

The detection unit 8 has a function to obtain a necessary signal or a reception echo from the signal processing unit 7 or the A/D converter 6, to perform envelope detection on the obtained pulse signal or reception echo, and to supply the detection result to the display unit 11 as a detection signal.

It may be come from behind placement of the signal processing unit 7 and the detection unit 8. In that case, the detection unit 8 obtains a necessary reception echo from the A/D converter 6, and performs envelope detection on the obtained reception echo. In addition, the signal processing unit 7 performs signal processing on the reception echo received from the detection unit 8.

The display unit 11 has a DSC (digital scan converter). The display unit 11 has a function to generate an image signal used for displaying an image on the monitor from the detection signal received from the detection unit 8, and to supply the generated image signal to the monitor 4 to cause the monitor 4 to display the image. The DSC of the display unit 11 is configured to convert the scan mode of the detection signal received from the detection unit 8 from an ultrasonic scan mode into a television scan mode for displaying.

The system control unit 10 has a function to entirely control each element in the apparatus main body 2 by supplying control signals to elements including the transmission and reception unit 5, the A/D converter 6, the signal processing unit 7, the detection unit 8, and the scan sequence control unit 9.

Then, an operation and action of the ultrasonic diagnostic apparatus 1 will be described below.

FIG. 2 is a flowchart showing a procedure for obtaining an ultrasonic diagnostic image of an object according to the contrast echo method using microbubbles as a contrast medium by the ultrasonic diagnostic apparatus 1 shown in FIG. 1. An alphabet “S” followed by a numeral in the figure represents each step of the flowchart.

Firstly, the contrast medium consisting of microbubbles is injected to the object. Consequently, many microbubbles are led to an imaging target part such as a blood vessel.

Then, in step S1, the first ultrasonic pulse P1 having the maximum amplitude in the positive pressure side is transmitted into the object and the first reception echo (echo 1) is acquired.

More specifically, the scan sequence control unit 9 generates a scan sequence so that the first ultrasonic pulse P1 having the maximum amplitude in the positive pressure side is transmitted from the ultrasonic probe 3. The scan sequence control unit 9 supplies the generated scan sequence to the transmission and reception unit 5. This causes the transmission and reception unit 5 to generate transmission signals according to the scan sequence received from the scan sequence control unit 9, and to supply the generated transmission signals to the respective ultrasonic transducers of the ultrasonic probe 3. Accordingly, the first ultrasonic pulses P1 each having the maximum amplitude in the positive pressure side are transmitted from the ultrasonic probe 3 to the imaging target part in the object.

Since many microbubbles exist in the imaging target part, the ultrasonic echoes, which are generated by reflection of the first ultrasonic pulses P1 on the microbubbles and tissues, are received by the ultrasonic probe 3. The ultrasonic echoes, corresponding to the first ultrasonic pulses P1, received by the ultrasonic probe 3 are converted into the first reception echoes (echo 1) which are electric signals and are supplied to the transmission and reception unit 5.

The transmission and reception unit 5 supplies the first reception echoes (echo 1) received from the ultrasonic probe 3 to the A/D converter 6. The A/D converter 6 converts the analog first reception echoes (echo 1) supplied from the transmission and reception unit 5 into the digital first reception echoes (echo 1). The A/D converter 6 supplies the digitalized first reception echoes (echo 1) to the signal processing unit 7.

The signal processing unit 7 performs predetermined processing including delay processing and phasing addition processing to the first reception echoes (echo 1) received from the A/D converter 6. The signal processing unit 7 then temporarily stores the first reception echo (echo 1) corresponding to the first ultrasonic pulse P1.

Then, in step S2, the second ultrasonic pulse P2 having the maximum amplitude in the negative pressure side is transmitted into the object and the second reception echo (echo 2) is acquired. Preferably, the time phase of the second ultrasonic pulse P2 is set to be different from that of the first ultrasonic pulse P1

Specifically, the second ultrasonic pulses P2 are transmitted into the object according to a flow similarly to that in case of transmission of the first ultrasonic pulses P1 and the second reception echo (echo 2) is obtained along a flow similarly to that in case of acquisition of the first reception echo (echo 1). The signal processing unit 7 then temporarily stores the second reception echo (echo 2) corresponding to the second ultrasonic pulse P2.

Then, in step S3, the third ultrasonic pulse P3 derived by a linear addition of the first ultrasonic pulse P1 and the second ultrasonic pulse P2 is transmitted into the object and the third reception echo (echo 3) is acquired.

Specifically, the third ultrasonic pulses P3 are transmitted into the object according to a flow similarly to that in case of transmission of the first ultrasonic pulses P1 and the third reception echo (echo 3) is obtained along a flow similarly to that in case of acquisition of the first reception echo (echo 1). The signal processing unit 7 then temporarily stores the third reception echo (echo 3) corresponding to the third ultrasonic pulse P3.

Then in step S4, for example as shown in as expression (1), a linear calculation is performed on the first reception echo (echo1), the second reception echo (echo2) and the third reception echo (echo3) by the signal processing unit 7. Thus, the first reception echo (echo1), the second reception echo (echo2) and the third reception echo (echo3) are compounded to the compound signal S.

S=echo1+echo2−echo3 (1)

Each of the first reception echo (echo1), the second reception echo (echo2) and the third reception echo (echo3) here contains linear component and nonlinear component from micro bubbles, and linear component and harmonic component, which is nonlinear component, from tissues. The maximum amplitude of the first ultrasonic pulse P1 to acquire the first reception echo (echo1) lies on the positive pressure side while the maximum amplitude of the second ultrasonic pulse P2 to acquire the second reception echo (echo2) lies on the negative pressure side. Moreover, the third ultrasonic pulse P3 to acquire the third reception echo (echo3) is obtained by a linear addition of the first ultrasonic pulse P1 and the second ultrasonic pulse P2.

In this instance, it is known that that micro bubbles respond more strongly, i.e., generate an ultrasonic echo having nonlinear component with a higher signal intensity to an ultrasonic wave having an amplitude in the negative pressure side than an ultrasonic wave having an amplitude in the positive pressure side.

Therefore, the respective nonlinear components of the first reception echo (echo1), the second reception echo (echo2) and the third reception echo (echo3) have mutual differences in signal intensities. On the other hand, an intensity of a signal derived by addition of the respective linear components of the first reception echo (echo1) and the second reception echo (echo2) is equivalent to a signal intensity of linear component of the third reception echo (echo3).

Consequently, as shown in expression (1), by adding the first reception echo (echo1) to the second reception echo (echo2) and subtracting the third reception echo (echo3) from the result of addition, the nonlinear component remains while the linear component is canceled out to be removed.

It is known that signal intensities of bubble echoes, which are echoes from microbubbles, of the nonlinear components are generally higher than those of the tissue echoes in the fundamental band. In addition, it is known that many nonlinear responses of microbubbles to ultrasonic waves exist in the fundamental band. On the other hands, it is known that the tissue echoes almost consist of linear components in the fundamental band while most harmonic components of the tissue echoes are in the second harmonic band.

Therefore, if the linear component is removed by a linear calculation, only a bubble echo that is nonlinear component from micro bubbles can be extracted in the fundamental wave band. So the compound signal S in the fundamental wave band can be used for imaging. For this purpose, the compound signal S in the fundamental wave band obtained by the linear calculation is provided from the signal processing unit 7 to the detection unit 8.

Note that, by setting the respective time phases of the maximum amplitudes of the first ultrasonic pulse P1 and the second ultrasonic pulse P2 so as to be mutually different, a difference between the respective signal intensities of the nonlinear components of the first reception echo (echo1) and the second reception echo (echo2) can become large to extract nonlinear component of a bubble echo showing a higher signal intensity. However, when a time phase difference between the respective maximum amplitudes of the first ultrasonic pulse P1 and the second ultrasonic pulse P2 is set be too large, a tissue echo might remain in the fundamental wave band by a linear calculation. Therefore, it is preferable that the time phase difference between the respective maximum amplitudes of the first ultrasonic pulse P1 and the second ultrasonic pulse P2 is set to an appropriate value obtained from a simulation or empirically.

Then, in step S5, the compound signal S in the fundamental band is imaged. For this purpose, the detection unit 8 performs envelope detection of the compound signal S and supplies the detection result to the display unit 11 as the detection signal. The display unit 11 then generates an imaging signal used for displaying an image on the monitor based on the detection signal received from the detection unit 8. The display unit 11 supplies the generated imaging signal to the monitor 4 to cause the monitor to display the image thereon.

Consequently, a contrast-enhanced image of the imaging target, such as blood vessel of the object which is enhanced by the contrast medium, is displayed on the monitor 4. Though this contrast-enhanced image is generated from the nonlinear components remaining in the fundamental band by the linear calculation, echoes from the tissues in the image are suppressed while echoes from bubbles are selectively used for generating the image.

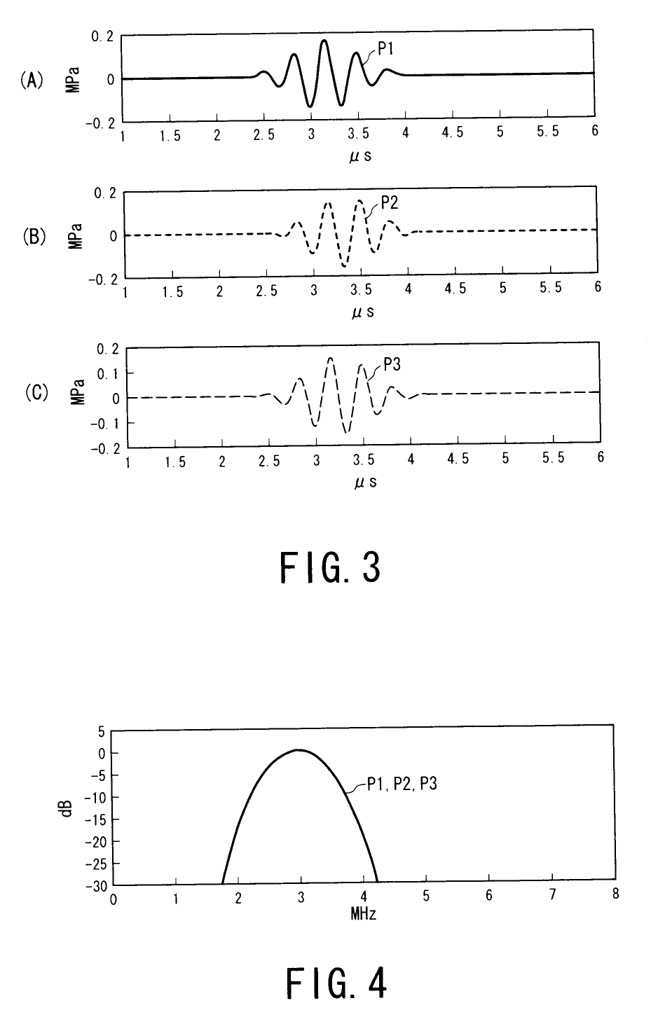

FIG. 3 is a diagram showing an example of setting the first ultrasonic pulse P1, the second ultrasonic pulse P2 and the third ultrasonic pulse P3 transmitted toward the object from the ultrasonic diagnostic apparatus 1 shown in FIG. 1 for a simulation.

In (A), (B) and (C) of FIG. 3, each of the respective abscissa axes denotes time (μs) and each of the respective ordinate axes denotes acoustic pressures (MPa). Further, FIG. 3 (A) shows the first ultrasonic pulse P1, FIG. 3 (B) shows the second ultrasonic pulse P2 and FIG. 3 (C) shows the third ultrasonic pulse P3. As shown in FIG. 3 (A), the first ultrasonic pulse P1 has the maximum amplitude in the positive pressure side. Further, as shown in FIG. 3 (B), the second ultrasonic pulse P2 has the maximum amplitude in the negative pressure side. A time phase of the maximum amplitude of the second ultrasonic pulse P2 is set to be different from that of the first ultrasonic pulse P1. Moreover, the third ultrasonic pulse P3 as shown in FIG. 3(C) can be generated by a linear addition of the first ultrasonic pulse P1 and the second ultrasonic pulse P2.

FIG. 4 is a diagram showing an example of a frequency spectrum of the first ultrasonic pulse P1, the second ultrasonic pulse P2 and the third ultrasonic pulse P3 each shown in FIG. 3.

In FIG. 4, the abscissa axis denotes frequency (MHz) and the ordinate axis denotes signal intensity level (dB). Further, the solid line in FIG. 4 shows the frequency spectrum of the first ultrasonic pulse P1, the second ultrasonic pulse P2 and the third ultrasonic pulse P3. As shown in FIG. 4, respective frequency spectra of the first ultrasonic pulse P1, the second ultrasonic pulse P2 and the third ultrasonic pulse P3 can be set to be mutually same.

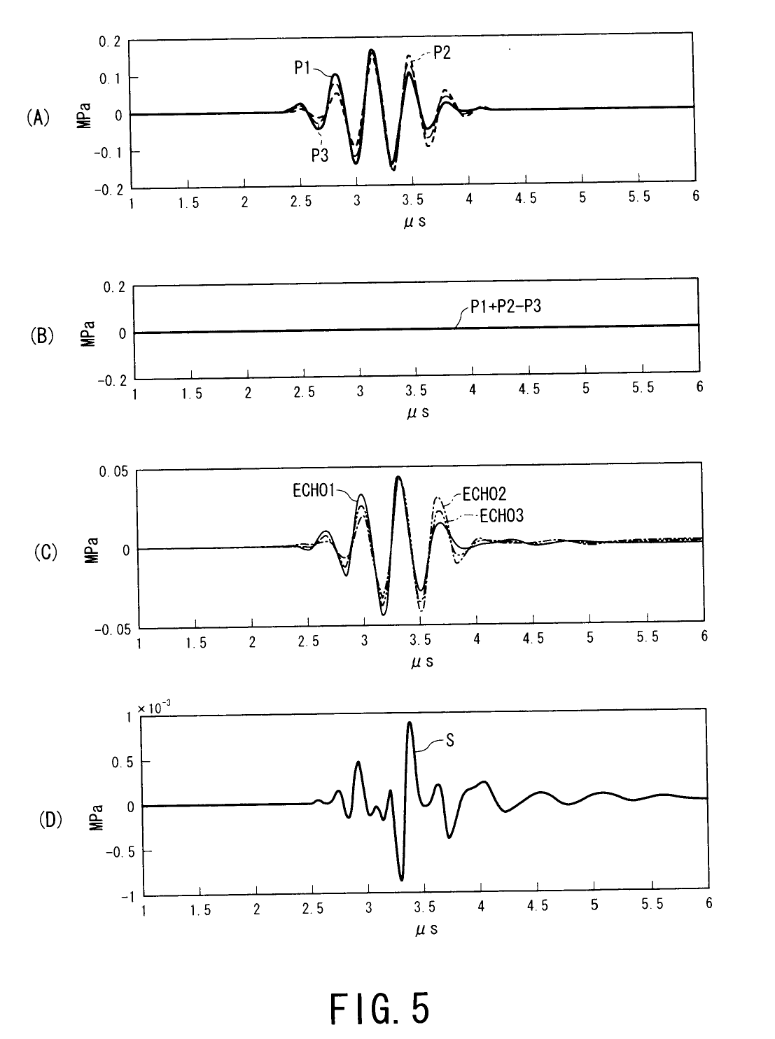

FIG. 5 is a diagram showing a compound signal S derived by a simulation using the first ultrasonic pulse P1, the second ultrasonic pulse P2 and the third ultrasonic pulse P3 shown in FIG. 3.

In (A), (B), (C) and (D) of FIG. 5, each abscissa axis denotes time (μs) and each ordinate axis denotes acoustic pressure (MPa). FIG. 5 (A) shows each waveform of the first ultrasonic pulse P1, the second ultrasonic pulse P2 and the third ultrasonic pulse P3. FIG. 5 (B) shows a result of addition of the first ultrasonic pulse P1 and the second ultrasonic pulse P2 and subtraction of the third ultrasonic pulse P3.

As shown in FIG. 5(B), since the third ultrasonic pulse P3 is a result of a linear addition of the first ultrasonic pulse P1 and the second ultrasonic pulse P2, a signal derived by adding the first ultrasonic pulse P1 to the second ultrasonic pulse P2 and subtracting the third ultrasonic pulse P3 from the additional result is canceled out to be zero. Therefore, a signal derived by adding linear component of the first reception echo (echo1) obtained due to transmission of the first ultrasonic pulse P1 to linear component of the second reception echo (echo2) obtained due to transmission of the second ultrasonic pulse P2 and subtracting the third reception echo (echo3) obtained due to transmission of the third ultrasonic pulse P3 from the additional result is also canceled out to be zero.

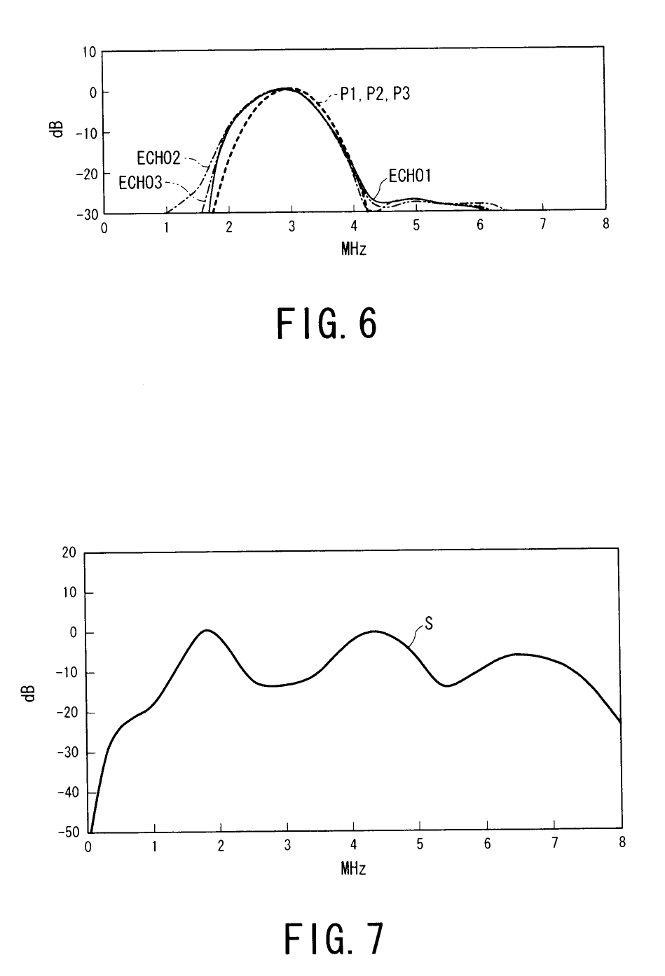

FIG. 5 (C) shows the first reception echo (echo1), the second reception echo (echo2) and the third reception echo (echo3) obtained by the simulation. Note that, a detail method for the simulation is described in “Experimental and Theoretical Evaluation of Microbubble Behavior: Effect of Transmitted Phase and Bubble Size”, Karen E. Morgan, John S. Allen, Paul A. Dayton, James E. Chomas, Alexander L. Klibanov and Katherine W. Ferrara, Member, IEEE, IEEE TRANSACTIONS ON ULTRASONICS, FERROELECTRICS, AND FREQUENCY CONTROL, VOL. 47, No. 6, NOVEMBER 2000. Further, main conditions are as follows. The sound velocity c=1540 (m/s). The transmission frequency f0=3 (MHz). The number of waves n=2. The thickness of shell F=1 (nm) FIG. 6 is a diagram showing each frequency spectrum of the first reception echo (echo1), the second reception echo (echo2) and the third reception echo (echo3) shown in FIG. 5 (C).

In FIG. 6, the abscissa axis denotes frequency (MHz) and the ordinate axis denotes signal intensity level (dB). Further, the dotted line in FIG. 6 shows the frequency spectrum of the first ultrasonic pulse P1, the second ultrasonic pulse P2 and the third ultrasonic pulse P3.

As shown in FIG. 6, the respective frequency spectra of the first reception echo (echo1), the second reception echo (echo2) and the third reception echo (echo3) become mutually different due to each nonlinear component. In addition, each frequency spectrum of the first reception echo (echo1), the second reception echo (echo2) and the third reception echo (echo3) also has a distribution different from the frequency spectrum of the first ultrasonic pulse P1, the second ultrasonic pulse P2 and the third ultrasonic pulse P3. In other words, it is recognized that the second reception echo (echo2) obtained by transmission of the second ultrasonic pulse P2 having the maximum amplitude in the negative pressure side has a signal distribution which distributes in a frequency band broader than a frequency band in which a signal distribution of the first reception echo (echo1) obtained by transmission of the first ultrasonic pulse P1 having the maximum amplitude in the positive pressure side distributes. This result is attributed to the fact that the second ultrasonic pulse P2 having the maximum amplitude in the negative pressure side responded more strongly to micro bubbles than the first ultrasonic pulse P1 having the maximum amplitude in the positive pressure side.

Since the maximum amplitude of the third ultrasonic pulse P3 or the pole corresponding to the maximum amplitude is different from that corresponding to each of the first ultrasonic pulse P1 and the second ultrasonic pulse P2, the third reception echo (echo3) obtained by transmission of the third ultrasonic pulse P3 also has a frequency spectrum different from that of each of the first reception echo (echo1) and the second reception echo (echo2).

Therefore, even if a linear calculation is performed on the first reception echo (echo1), the second reception echo (echo2) and the third reception echo (echo3) as shown in the expression (1), nonlinear component showing a sufficiently high signal intensity remains as the compound signal S.

FIG. 5 (D) shows the compound signal S derived by the linear calculation of the first reception echo (echo1), the second reception echo (echo2) and the third reception echo (echo3). That is to say, adding the first reception echo (echo1) to the second reception echo (echo2) and subtracting the third reception echo (echo3) can generate the compound signal S shown in FIG. 5(D). In other words, unlike the result of the linear calculation to the linear components as shown in FIG. 5(B), nonlinear component of a bubble echo is not cancelled by the linear calculation and the compound signal S shown in FIG. 5(D) remains.

FIG. 7 is a diagram showing the frequency spectrum of the compound signal S shown in FIG. 5 (D).

In FIG. 7, the abscissa axis denotes frequency (MHz) and the ordinate axis denotes signal intensity level (dB). In the fundamental wave band, the compound signal S having the frequency spectrum as shown in FIG. 7 contains little harmonic component of a tissue echo. Consequently, imaging the compound signal S in the fundamental wave band can obtain an ultrasonic diagnostic image corresponding to bubble echos with a reduced tissue echo in a satisfactory contrast ratio.

By the way, the first ultrasonic pulse P1, the second ultrasonic pulse P2 and the third ultrasonic pulse P3 can be transmitted with setting the amplitudes, the phases, the center frequencies and the frequency bands thereof into desired values by controlling the transmission and reception unit 5 under the scan sequence control unit 9.

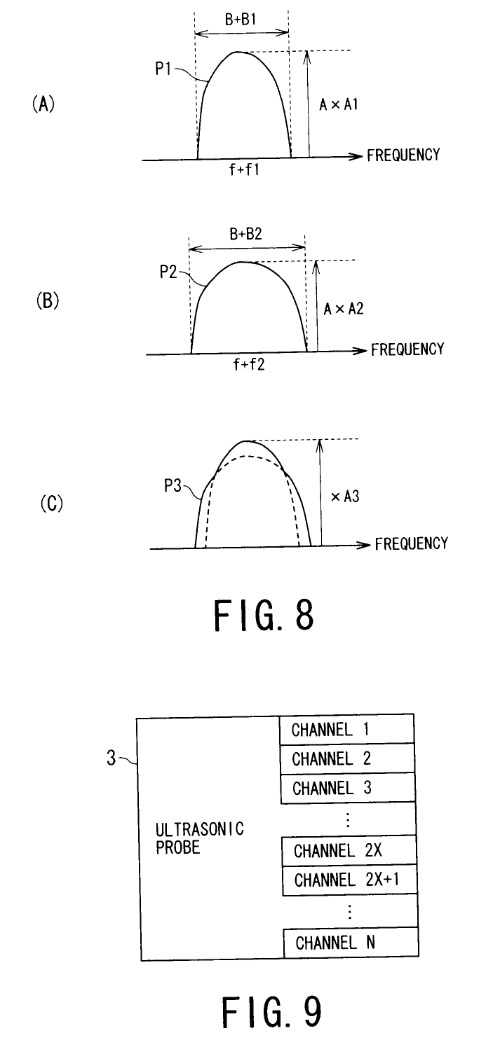

FIG. 8 is a diagram showing an example case of transmitting the first ultrasonic pulse P1, the second ultrasonic pulse P2 and the third ultrasonic pulse P3 from the ultrasonic diagnostic apparatus 1 shown in FIG. 1 with setting the amplitudes, the phases, the center frequencies and the frequency bands thereof into desired values.

In (A), (B) and (C) of FIG. 8, each abscissa axis denotes frequency. Further, FIG. 8 (A) shows the first ultrasonic pulse P1, FIG. 8 (B) shows the second ultrasonic pulse (B) and FIG. 8 (C) shows the third ultrasonic pulse P3.

As shown in FIG. 8(A), an amplitude, a phase, a center frequency and/or a frequency band of the first ultrasonic pulse P1 can be set to a value derived by multiplying a reference amplitude A by A1, a value derived by increasing by φ1 (deg) from a reference phase φ, a value derived by increasing by f1 from a reference center frequency f and/or a value derived by increasing by B1 from a reference frequency band B, respectively. Similarly, an amplitude, a phase, a center frequency and/or a frequency band of the second ultrasonic pulse P2 can be set to a value derived by multiplying the reference amplitude A by A2, a value derived by increasing by φ2 (deg) from the reference phase φ, a value derived by increasing by f2 from the reference center frequency f and/or a value derived by increasing by B2 from the reference frequency band B, respectively, as shown in FIG. 8(B).

By a linear addition of the first ultrasonic pulse P1 shown in FIG. 8(A) and the second ultrasonic pulse P2 shown in FIG. 8(B), multiplying an amplitude by A3 and inverting a phase by +180 degrees, the third ultrasonic pulse P3 as shown in FIG. 8(C) can be generated.

Transmitting the first ultrasonic pulse P1, the second ultrasonic pulse P2 and the third ultrasonic pulse P3 as shown in FIG. 8(A), (B), (C) respectively can obtain corresponding echoes of the first reception echo (echo1), the second reception echo (echo2) and the third reception echo (echo3). In this case, in the signal processing unit 7, adding the first reception echo (echo1), the second reception echo (echo2) and a signal derived by multiplying a signal intensity of the third reception echo (echo3) by 1/A3 can cancel linear component to extract only nonlinear component. Then, a tissue echo is removed and imaging can be performed by using nonlinear component of a bubble echo selectively.

Thus, transmission parameters including amplitude, a phase, a center frequency and a frequency band of each of the first ultrasonic pulse P1, the second ultrasonic pulse P2 and the third ultrasonic pulse P3 can be set to desired values. In that case, a linear calculation method is determined according to the transmission parameters so that nonlinear component can be extracted. Therefore, correction of not only each amplitude of the first ultrasonic pulse P1, the second ultrasonic pulse P2 and the third ultrasonic pulse P3 but also each phase thereof can also be performed in the linear calculation.

When the first ultrasonic pulse P1, the second ultrasonic pulse P2 and the third ultrasonic pulse P3 are transmitted with mutually different transmission parameters respectively, a difference among the respective signal intensities of nonlinear components of the bubble echoes corresponding to the first reception echo (echo1), the second reception echo (echo2) and the third reception echo (echo3) can be expected to become larger. For this purpose, the optimum transmission parameters can be obtained in advance by a simulation or an imaging test.

So far, a case was explained as an example where transmission waveform of the third ultrasonic pulse P3 was obtained electronically by controlling the transmission and reception unit 5 so as to perform compound processing of the first ultrasonic pulse P1 and the second ultrasonic pulse P2 with the scan sequence control unit 9. However, the transmission waveform of the third ultrasonic pulse P3 can be generated as a transmission acoustic field without controlling the transmission and reception unit 5.

To improve suppression effect of a tissue echo, it is important to generate a waveform of compounded pulse such as the third ultrasonic pulse P3 with a high accuracy and transmit it. However, performance of a pulsar included in the transmission and reception unit 5 in order to apply a transmission pulse to the ultrasonic probe 3 might be insufficient for generating the waveform of the compounded pulse with a high accuracy. For that reason, plural transmission pulses, before composition, for generating a compounded pulse such as the third ultrasonic pulse P3 can be transmitted sequentially from mutually different transmission apertures while the compounded pulse formed as a transmission sound field can be transmitted by simultaneously transmitting the plural pulses to be compounded from the respective transmission apertures.

FIG. 9 is a diagram showing channels formed by the plural ultrasonic transducers included in the ultrasonic probe 3 shown in FIG. 1.

As shown in FIG. 9, the ultrasonic probe 3 has n pieces of transmitting and receiving channels for transmission of an ultrasonic pulse and reception of an ultrasonic echo. Each channel can be assigned so that all or a part of multiple channels for transmitting the first ultrasonic pulse P1 are different from channels for transmitting the second ultrasonic pulse P2. When the first ultrasonic pulse P1 and the second ultrasonic pulse P2 are transmitted simultaneously by using both channels used for transmitting the first ultrasonic pulse P1 and the second ultrasonic pulse P2 respectively, the third ultrasonic pulse P3 can be generated as a transmission acoustic field.

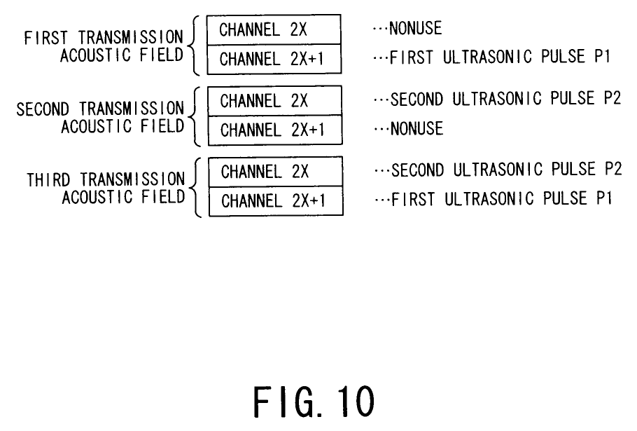

FIG. 10 is a diagram showing an example case of synthesizing the third ultrasonic pulses P3 as a transmission acoustic field by transmitting the first ultrasonic pulses P1 and the second ultrasonic pulses P2 from mutually different and exclusive plural channels of the ultrasonic probe 3 shown in FIG. 9.

As shown in FIG. 10, an ultrasonic pulse is transmitted three times sequentially, and consequently, the first transmission acoustic field, the second transmission acoustic field and the third transmission acoustic field are formed respectively. The first transmission acoustic field is formed by transmitting the first ultrasonic pulse P1 from the odd channels (CHANNEL 2X+1), which are exclusive against the even channels, of the ultrasonic probe 3 without using the even channels (CHANNEL 2X) of the ultrasonic probe 3. The second transmission acoustic field is formed by transmitting the second ultrasonic pulse P2 from the even channels (CHANNEL 2X) without using the odd channels (CHANNEL 2X+1). The third transmission acoustic field is formed by simultaneously transmitting the first ultrasonic pulse P1 from the odd channels (CHANNEL 2X+1) and the second ultrasonic pulse P2 from the even channels (CHANNEL 2X) respectively.

In this way, by forming a waveform of an ultrasonic pulse to be composed as a transmission acoustic field without electronic control, a compound ultrasonic pulse with a high-accuracy waveform can be transmitted and suppression effect of tissue echo can be improved further.

As described above, the ultrasonic diagnostic apparatus 1 is an apparatus which obtains three reception echoes by transmitting both of two ultrasonic pulses having the maximum amplitudes in the positive and negative pressure sides respectively and a pulse derived by compounding the two ultrasonic pulses, and extracts nonlinear component of a bubble echo by a linear calculation of the obtained reception echo signals to be imaged. That is, by using the property that micro bubbles show mutually different responses to transmission of two ultrasonic pulses having the maximum amplitudes in the positive and negative pressure sides respectively, nonlinear component of a bubble echo is extracted by a linear calculation.

Consequently, according to the ultrasonic diagnostic apparatus 1, nonlinear component of a bubble echo can be extracted efficiently by a linear calculation of reception echo signals to plural ultrasonic pulses having mutually different waveforms. In addition, a tissue echo can be reduced since the fundamental wave band containing no second harmonic component of the tissue echo is used for imaging. As a result, a contrast of signals from bubbles with regard to signals from a tissue can be improved.

What is claimed is:

1. An ultrasonic diagnostic apparatus comprising:
an ultrasonic transmission/reception unit configured to transmit a first ultrasonic pulse having a maximum amplitude in a positive pressure, a second ultrasonic pulse having a maximum amplitude in a negative pressure and a third ultrasonic pulse derived by compounding the first ultrasonic pulse with the second ultrasonic pulse into an object in which a contrast medium is injected and obtain a first reception echo, a second reception echo and a third reception echo corresponding to the first ultrasonic pulse, the second ultrasonic pulse and the third ultrasonic pulse respectively; and
an image generating unit configured to generate a compounded signal which is a nonlinear component from the contrast medium by a linear calculation of the first reception echo, the second reception echo and the third reception echo and generate image data of the object by using the compounded signal.

2. An ultrasonic diagnostic apparatus of claim 1,
wherein said image generating unit is configured to generate the image data by using compounded signal in a fundamental wave band.

3. An ultrasonic diagnostic apparatus of claim 1,
wherein said ultrasonic transmission/reception unit is configured to set at least one of respective amplitudes, respective phases, respective center frequencies and respective frequency bands of the first ultrasonic pulse and the second ultrasonic pulse to be mutually different values.

4. An ultrasonic diagnostic apparatus of claim 1,
wherein said ultrasonic transmission/reception unit is configured to form the third ultrasonic pulse as a transmission sound field by transmitting the first ultrasonic pulse and the second ultrasonic pulse from plural channels of which at least a part for transmitting the first ultrasonic pulse is different from channels for transmitting the second ultrasonic pulse.

5. An ultrasonic diagnostic apparatus of claim 1,
wherein said ultrasonic transmission/reception unit is configured to transmit the first ultrasonic pulse and the second ultrasonic pulse at mutually different time phases.

6. An ultrasonic diagnostic apparatus of claim 1,
wherein said image generating unit is configured to correct at least one of amplitudes and phases of the first reception echo, the second reception echo and the third reception echo on the linear calculation.

7. An ultrasonic diagnostic method comprising:
transmitting a first ultrasonic pulse having a maximum amplitude in a positive pressure, a second ultrasonic pulse having a maximum amplitude in a negative pressure and a third ultrasonic pulse derived by compounding the first ultrasonic pulse with the second ultrasonic pulse into an object in which a contrast medium is injected and obtaining a first reception echo, a second reception echo and a third reception echo corresponding to the first ultrasonic pulse, the second ultrasonic pulse and the third ultrasonic pulse respectively; and
generating a compounded signal which is a nonlinear component from the contrast medium by a linear calculation of the first reception echo, the second reception echo and the third reception echo and generating image data of the object by using the compounded signal.

8. An ultrasonic diagnostic method of claim 7,
wherein the image data is generated by using compounded signal in a fundamental wave band. said image generating unit is configured to

9. An ultrasonic diagnostic method of claim 7,
wherein at least one of respective amplitudes, respective phases, respective center frequencies and respective frequency bands of the first ultrasonic pulse and the second ultrasonic pulse is set to be mutually different values.

10. An ultrasonic diagnostic method of claim 7,
wherein the third ultrasonic pulse is formed as a transmission sound field by transmitting the first ultrasonic pulse and the second ultrasonic pulse from plural channels of which at least a part for transmitting the first ultrasonic pulse is different from channels for transmitting the second ultrasonic pulse.

11. An ultrasonic diagnostic method of claim 7,
wherein the first ultrasonic pulse and the second ultrasonic pulse are transmitted at mutually different time phases.

12. An ultrasonic diagnostic method of claim 7,
wherein at least one of amplitudes and phases of the first reception echo, the second reception echo and the third reception echo are corrected on the linear calculation.
